(19) United States
(12) Patent Application Publication
(10) Pub. No.: US 2026/0351410 A1
(43) Pub. Date: Nov. 5, 2026

(54) SYSTEM AND METHOD FOR RARE DISEASE DIFFERENTIAL DIAGNOSIS USING MULTI-MODEL CONSENSUS AGGREGATION WITH RECIPROCAL RANK FUSION

(71) Applicant: Chandrashekhar Sattiraju, Westwood, MA (US)

(72) Inventor: Chandrashekhar Sattiraju, Westwood, MA (US)

(21) Appl. No.: 19/630,432

(22) Filed: Mar. 27, 2026

Related U.S. Application Data
(63) Continuation-in-part of application No. 19/197,970, filed on May 3, 2025.

(51) Int. Cl. G16H 50/20

(52) U.S. Cl. CPC G16H 50/20

(57) ABSTRACT

A computer-implemented system for rare disease differential diagnosis employs a heterogeneous multi-model consensus architecture. Patient phenotypic input is normalized to OBSERVED and EXCLUDED Human Phenotype Ontology identifier sets. A pathway orchestration module concurrently dispatches the phenotype profile to independent diagnostic pathways—Bayesian ranking, LLM cross-validation, frequency-weighted negation-aware ontology matching, genetic variant prioritization, and community intelligence validation—each executing under read-isolation. Candidate diseases identified by name are resolved through multi-tier matching; unresolvable candidates are excluded before aggregation. Frequency-weighted specificity scoring bypasses Jaccard normalization bias via an unnormalized additive bonus. Negation-aware scoring penalizes candidates whose profiles include features reported as absent. A consensus aggregation engine implements Reciprocal Rank Fusion with a consensus bonus. A bounded self-reflective evaluation subsystem with Bayesian protection constraints resolves ambiguous candidates. A clinician confirmation gate gates patient-facing output.

### CROSS-REFERENCE TO RELATED APPLICATIONS

This application is a continuation-in-part of U.S. Non-Provisional Utility Patent Application No. Ser. No. 19/197,970, filed May 3, 2025, entitled “RARE DISEASE DIAGNOSIS AND ACCESS INTEGRATED PLATFORM,” the entire disclosure of which is incorporated herein by reference. The aforementioned application claims priority to U.S. Provisional Application Nos. 63/786,308; 63/791,504; 63/791,511; 63/791,514; 63/791,517; 63/791,520; 63/791,489; 63/795,358; 63/795,360; and 63/795,362, each filed in 2025, each incorporated herein by reference in its entirety.

### STATEMENT REGARDING FEDERALLY SPONSORED RESEARCH

Not applicable.

### FIELD OF THE INVENTION

The present invention relates to computer-implemented systems and methods for supporting rare disease differential diagnosis, and more particularly to a multi-model consensus diagnostic architecture in which a plurality of heterogeneous, independently executing diagnostic pathway modules concurrently process standardized phenotype codes under read-isolated parallel dispatch and contribute pathway-specific ranked outputs that are aggregated using a Reciprocal Rank Fusion algorithm augmented by a consensus bonus mechanism, negation-aware phenotype scoring, frequency-weighted information content specificity optimization, null-identifier filtering of unresolvable disease names, and a bounded self-reflective evaluation loop with Bayesian protection constraints, to produce a confidence-calibrated ranked differential diagnosis for clinician review.

### BACKGROUND OF THE INVENTION

Rare disease diagnosis presents a distinctive challenge in medical informatics. Approximately 7,000 to 10,000 known rare diseases collectively affect over 300 million individuals worldwide. The average diagnostic delay from symptom onset to confirmed diagnosis exceeds 4.8 years, during which patients receive an average of three incorrect diagnoses and accumulate significant medical costs. The root cause of this diagnostic gap is not merely physician inexperience; it is the fundamental computational challenge of differentiating among thousands of diseases sharing overlapping phenotypic profiles from limited patient-reported symptom data.

Existing computer-implemented diagnostic decision support systems suffer from a fundamental architectural limitation: they typically rely on a single algorithmic model or a single analytical pathway to generate diagnostic recommendations. Single-model systems exhibit characteristic failure modes. Bayesian phenotype-ranking algorithms, such as LIRICAL, exhibit high precision for diseases with well-characterized phenotype frequency distributions but underperform for atypical presentations and diseases with sparse annotation data. Large language model (LLM)-based diagnostic systems achieve broad coverage but may produce disease names that do not correspond to recognized ontological identifiers, generating outputs that cannot be matched against structured disease databases. Ontology-matching systems based on information content-weighted term similarity perform well when patient-reported HPO terms precisely match annotated disease terms but fail when lay-language symptom descriptions introduce normalization variability.

A critical limitation of existing ontology-matching systems is their reliance on normalized Jaccard-type denominators when computing phenotypic similarity. The standard SimGIC formulation computes similarity as the ratio of information content (IC) in the intersection of query and disease term sets to the IC in their union. This normalization penalizes diseases with large annotation sets relative to the query, creating a systematic bias against well-annotated diseases. To the applicant's knowledge, existing systems do not address this bias through a frequency-weighted specificity optimization in which disease-phenotype frequency annotations drawn from curated databases are used to weight each term's contribution to the similarity score as an unnormalized additive bonus, enabling rare but highly specific symptoms to exert disproportionate influence on the ranking while bypassing the denominator-driven bias inherent in standard formulations.

A further limitation is that existing systems generally do not adequately distinguish between observed clinical features and explicitly excluded clinical features. When a clinician or patient reports that a patient does NOT exhibit a particular symptom or sign, this negated feature is diagnostically informative: it penalizes diseases for which that feature is obligate or highly characteristic. While some prior systems have explored negated phenotype terms, existing approaches generally do not apply negation-aware scoring in which excluded Human Phenotype Ontology (HPO) features actively penalize candidate diseases proportional to the information content of the excluded feature within the disease's phenotype profile.

Additionally, existing systems generally do not combine the outputs of multiple heterogeneous diagnostic pathways through a mathematically principled rank aggregation method that is demonstrably more robust than weighted score combination when pathway scores are not calibrated to a common scale. Reciprocal Rank Fusion (RRF), as described by Cormack, Clarke, and Buettcher (SIGIR 2009), achieves this property by operating on rank positions rather than raw scores, making it resistant to inter-pathway score calibration differences that would otherwise produce artifacts in weighted score combination.

Existing self-reflective evaluation mechanisms in diagnostic AI systems are generally limited to single-pass evaluation. Existing systems generally do not employ a bounded self-reflective evaluation loop in which a large language model performs structured multi-step candidate reevaluation—assessing positive feature support, negative feature contradiction, and guideline-based diagnostic criteria matching—and then emits a bounded score adjustment that is constrained to protect high-confidence probabilistic signals from override.

Furthermore, existing systems generally do not incorporate a disease characterization richness prior derived from external genomic databases (such as Clin Var gene-disease association counts) to boost the ranking of diseases whose genomic underpinning is well-established, a signal that correlates with diagnostic accuracy because well-characterized diseases have more precise phenotype annotations.

There exists a need in the art for a computer-implemented rare disease diagnostic support system that: (i) enforces strict pathway independence through read-isolated parallel execution to maximize the information gain from multi-pathway combination; (ii) applies negation-aware phenotype scoring to use excluded features as diagnostic discriminators; (iii) implements frequency-weighted information content specificity scoring that bypasses Jaccard normalization bias via an unnormalized additive bonus; (iv) aggregates pathway outputs through Reciprocal Rank Fusion rather than calibrated score weighting; (v) filters unresolvable disease names generated by language model pathways before rank aggregation; (vi) applies a consensus bonus that rewards inter-pathway agreement while maintaining the independence of each pathway's contribution; (vii) employs a bounded self-reflective evaluation loop with explicit adjustment constraints that protect high-confidence Bayesian signals from override; and (viii) incorporates disease characterization richness priors from external genomic databases.

### SUMMARY OF THE INVENTION

The present invention provides a computer-implemented system and method for rare disease differential diagnosis support employing a heterogeneous multi-model consensus aggregation architecture with Reciprocal Rank Fusion. The invention achieves specific technical improvements over prior art diagnostic systems by: (a) enforcing pathway independence through asynchronous parallel execution with read-isolation, preventing inter-pathway correlation that would reduce consensus information gain; (b) implementing negation-aware information content-weighted phenotype similarity scoring in which excluded HPO features impose a penalty proportional to their information content within the candidate disease's annotated phenotype profile; (c) implementing frequency-weighted specificity scoring that assigns each HPO term a contribution weight derived from curated disease-phenotype frequency annotations, enabling rare pathognomonic features to exert disproportionate influence on the similarity score while bypassing the normalizing denominator bias inherent in standard SimGIC formulations; (d) applying Reciprocal Rank Fusion with a configurable k parameter to aggregate rank-ordered pathway outputs in a manner resistant to inter-pathway score calibration differences; (e) filtering candidate diseases whose names cannot be resolved to recognized identifiers before rank aggregation, preventing unresolvable outputs from corrupting the consensus; (f) applying a consensus bonus to candidate diseases receiving affirmative scores from a configurable threshold number of independent pathways; (g) employing a bounded self-reflective evaluation loop in which a large language model evaluation instance performs structured candidate reevaluation—assessing observed-feature support, excluded-feature contradiction, and guideline-based diagnostic criteria matching—with bounded adjustment constraints that protect high-confidence Bayesian signals from LLM-based override; and (h) incorporating a disease characterization richness prior derived from external gene-disease association databases to boost the ranking of genomically well-characterized diseases.

In one embodiment, the plurality of pathways comprises: (a) a Bayesian phenotype-ranking pathway applying a Bayesian probabilistic rare disease ranking algorithm; (b) an LLM cross-validation pathway executing a large language model under constrained inference parameters with structured output schema enforcement; (c) a direct ontology-matching pathway computing negation-aware, frequency-weighted information content-weighted phenotypic similarity against a rare disease ontology database; (d) a genetic variant prioritization pathway conditionally activated when genomic data is present; and (e) a community intelligence validation pathway cross-referencing candidate diseases against consented, anonymized community phenotype signals. Each pathway executes independently without receiving intermediate results from any other pathway.

The system further provides a bounded self-reflective diagnostic evaluation loop in which a large language model evaluation instance critically reevaluates top-ranked candidate diseases through structured multi-step analysis, assessing whether the patient's observed features support the candidate, whether excluded features contradict the candidate, whether diagnostic criteria from published clinical guidelines that are stored in a versioned guideline reference accessible to the evaluation instance are met, and whether phenotypic overlap between competing top-ranked candidates provides differential discrimination. The evaluation output applies bounded score adjustments that are constrained to prevent demotion of high-confidence Bayesian pathway signals. A clinician confirmation gate requires explicit clinician acknowledgment before any ranked output is communicated to patients.

### BRIEF DESCRIPTION OF THE DRAWINGS

FIG. 1 is a block diagram of the multi-model consensus diagnostic system 100 architecture showing the symptom intake module 110, phenotype normalization engine 120, pathway orchestration module 130, pathway modules 140a-140e, consensus aggregation engine 150, self-reflective evaluation subsystem 155 with bounded adjustment capability, confidence calibration module 160, and clinician output module 170.

FIG. 2 is a flowchart illustrating the parallel execution of diagnostic pathways, the null-identifier filtering step, the Reciprocal Rank Fusion aggregation process, the consensus bonus computation, and the bounded self-reflective evaluation loop.

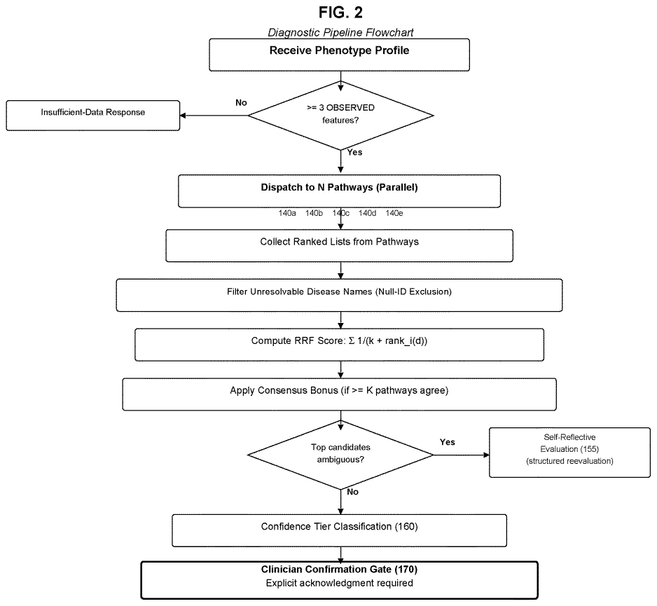

FIG. 3 is a data flow diagram illustrating negation-aware, frequency-weighted SimGIC scoring, showing the computation of specificity-weighted positive similarity, penalty scores for excluded HPO features, and their integration into the final phenotypic similarity score.

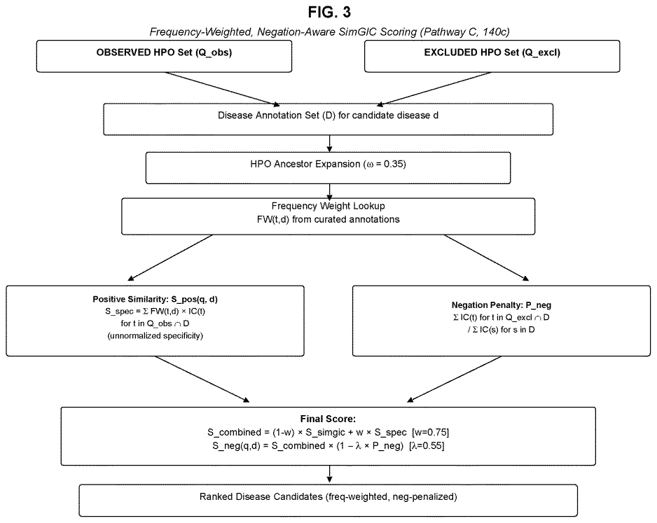

FIG. 4 is a diagram illustrating confidence tier classification thresholds, the insufficient-data detection path, and the clinician confirmation gate.

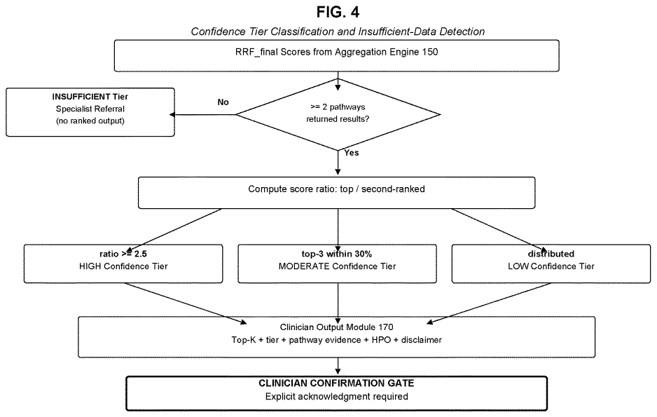

FIG. 5 is a data flow diagram illustrating the frequency-weighted information content specificity optimization, showing how disease-phenotype frequency annotations derived from curated databases modify the standard SimGIC computation.

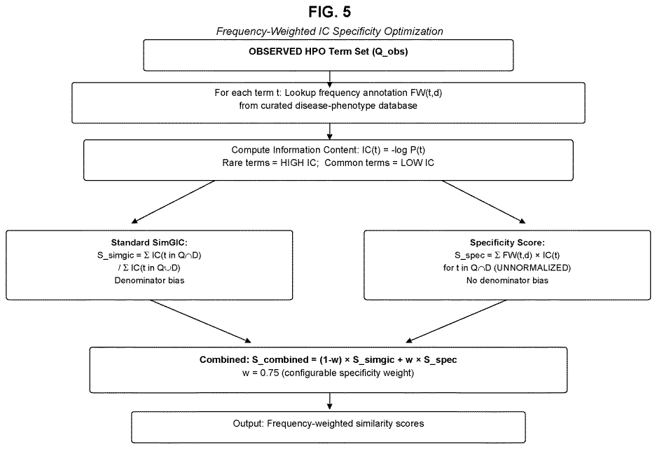

FIG. 6 is a block diagram illustrating the self-reflective evaluation subsystem 155, showing the structured multi-step candidate reevaluation process, bounded adjustment constraints, and Bayesian protection mechanism.

### DETAILED DESCRIPTION OF THE PREFERRED EMBODIMENTS

The following description sets forth specific details such as particular embodiments, procedures, techniques, and the like for purposes of explanation and not limitation. It will be appreciated by one skilled in the art that other embodiments may be employed apart from these specific details.

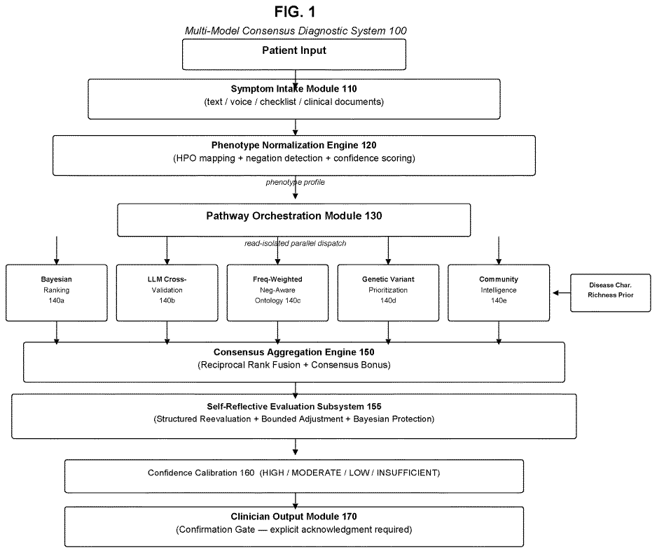

Referring to FIG. 1, the multi-model consensus diagnostic system 100 comprises: a symptom intake module 110; a phenotype normalization engine 120; a pathway orchestration module 130; diagnostic pathway modules 140a through 140e; a consensus aggregation engine 150 implementing Reciprocal Rank Fusion; a self-reflective evaluation subsystem 155 with bounded adjustment and Bayesian protection capability; a confidence calibration module 160; and a clinician output module 170.

The symptom intake module 110 receives patient symptom input in any of the following modalities: typed natural language text; structured checklist selections mapped to HPO term identifiers; voice transcription output from a speech-to-text engine configured with medical vocabulary models; structured clinical documents including clinical notes, laboratory reports, and genetic testing reports; or multi-modal input combining two or more of the foregoing modalities. The module applies completeness validation requiring a minimum of three observed phenotypic features before initiating the full multi-pathway diagnostic pipeline. When fewer than three observed features are present, the module returns an insufficient-data response with specialist referral recommendations without executing the full diagnostic pathway ensemble.

The phenotype normalization engine 120 maps received symptom input to Human Phenotype Ontology (HPO) term identifiers. The mapping process applies: (i) exact-match lookup against a curated lay-to-HPO synonym lexicon comprising at least 50,000 synonym entries; (ii) approximate-match retrieval using edit-distance metrics for typographic variants with a configurable similarity threshold; (iii) negation detection using grammatical pattern analysis to identify features explicitly reported as absent, classifying each HPO term as either OBSERVED or EXCLUDED; (iv) temporal qualifier extraction classifying features as current, historical, or intermittent; and (v) confidence scoring for each mapped term reflecting the certainty of the natural-language-to-HPO-term mapping. The output of the normalization engine is a structured phenotype profile comprising an OBSERVED set and an EXCLUDED set of HPO term identifiers with associated confidence scores.

A critical architectural requirement governing the operation of pathway modules 140a through 140e is pathway independence. Each pathway module receives only the phenotype profile from the phenotype normalization engine 120 and produces only a ranked list of candidate diseases with associated scores. No pathway module receives or accesses intermediate results, ranked outputs, or probability scores from any other pathway module during execution. The pathway orchestration module 130 enforces independence by dispatching the phenotype profile to all pathway modules simultaneously via asynchronous parallel execution with read-isolation, ensuring no pathway can access another pathway's computational state. This independence constraint is a prerequisite for valid Reciprocal Rank Fusion, as correlation between pathway outputs would reduce the effective information gain from multi-pathway combination.

Pathway A—Bayesian Phenotype-Ranking Pathway 140a: This pathway applies a Bayesian probabilistic ranking algorithm, such as LIRICAL (Likelihood Ratio Interpretation of Clinical Abnormalities) or an equivalent peer-reviewed Bayesian rare disease ranking system, to the OBSERVED HPO terms in the phenotype profile. The pathway queries a local rare disease knowledge base comprising disease-phenotype frequency annotations for at least 7,800 rare diseases, computes log-likelihood ratio scores for each candidate disease, and produces a ranked list with posterior probability estimates.

Pathway B—LLM Cross-Validation Pathway 140b: This pathway executes an independent large language model (LLM) inference pass under constrained parameters comprising: a locked model version identifier to ensure deterministic and reproducible behavior across evaluation sessions; a constrained output schema enforcing structured JSON with disease name, confidence, and reasoning fields; temperature parameter set to zero for deterministic inference; and explicit system prompt prohibition on free-text diagnostic language in outputs. The LLM receives the phenotype profile expressed as a list of HPO term labels and generates a ranked disease candidate list. Extracted disease names are matched against a disease name index using a multi-tier fuzzy matching procedure comprising: (i) exact normalized-name lookup after case folding and whitespace normalization; (ii) edit-distance-based approximate matching with a configurable similarity threshold (default: Levenshtein ratio >=0.85); and (iii) word-overlap Jaccard similarity scoring for multi-word disease names. When a candidate disease cannot be matched to a recognized disease identifier through any tier of matching, it is recorded with a null identifier and excluded from RRF aggregation, preventing unresolvable disease names from affecting the consensus ranking.

Pathway C—Negation-Aware, Frequency-Weighted Ontology-Matching Pathway 140c: This pathway computes a frequency-weighted, negation-aware information content phenotypic similarity score for each candidate disease using the following procedure. For each candidate disease d with annotated HPO term set D: (i) For each HPO term t, its information content IC(t) is computed as the negative log probability of the term's occurrence frequency across all disease annotations in the phenotype database. A frequency weight FW(t,d) is derived from curated disease-phenotype frequency annotations (when available) reflecting how frequently term t is observed in patients with disease d, with higher frequency annotations receiving proportionally higher weights. Terms annotated as “obligate” or “very frequent” receive maximum frequency weight; terms annotated as “occasional” or “excluded” receive reduced or zero frequency weight. (ii) The positive similarity component S_pos(q,d) is computed using a frequency-weighted SimGIC variant: S_pos(q,d)=Σ(FW(t,d)×IC(t) for t in Q_obs∩D)/Σ(IC(t) for t in Q_obs∪D). (iii) In an alternative embodiment achieving superior empirical performance, the frequency-weighted specificity component is computed as an additive bonus rather than a multiplicative modification: S_spec(q,d)=Σ(FW(t,d)×IC(t) for t in Q_obs∩D), wherein the sum is not divided by a union-based denominator, thereby bypassing the Jaccard normalization that would otherwise penalize well-annotated diseases with large annotation sets. In this embodiment, the specificity score is combined with the standard SimGIC score via a configurable weighting parameter w (default w=0.75): S_combined(q,d)=(1−w)×S_simgic(q,d)+w×S_spec(q,d). (iv) The negation penalty component P_neg(q_excl,d) is computed for excluded features: for each term t in the EXCLUDED set Q_excl that is also present in D, a penalty proportional to IC(t)/Σ(IC(s) for s in D) is computed. (v) The final negation-aware, frequency-weighted score is S_neg(q,d)=S_combined(q,d)×(1−λ×P_neg(q_excl,d)), where λ is a configurable penalty weight parameter with default value 0.55. (vi) HPO ancestor terms from the ontology hierarchy are included in the comparison with a reduced weight parameter ω (default 0.35) to capture ontological proximity when specific terms are not present in the disease annotation database.

Pathway D—Genetic Variant Prioritization Pathway 140d: This pathway is conditionally activated when genomic variant data is present in the patient input. When activated, the pathway: (i) cross-references reported variants against curated variant-disease association databases including Clin Var, OMIM variant tables, and equivalent resources; (ii) applies variant pathogenicity classification scoring, weighting pathogenic and likely pathogenic variants higher than variants of uncertain significance; (iii) applies inheritance pattern filtering, requiring homozygous or compound heterozygous variants for autosomal recessive diseases and heterozygous variants for autosomal dominant diseases; (iv) applies variant type scoring that differentiates between loss-of-function variants (including nonsense, frameshift, canonical splice-site, and large deletion variants) and missense variants, wherein loss-of-function variants receive a higher pathogenicity weight because they are more likely to ablate gene function entirely, while missense variants receive a reduced weight reflecting their variable functional impact, with the specific weight differential being configurable (default: loss-of-function weight=1.0, missense weight=0.6, variant of uncertain significance weight=0.2); and (v) computes a genetic evidence score for each candidate disease. When not activated due to absence of genomic input, this pathway contributes a defined null score to the consensus aggregation that does not penalize diseases for lack of genetic confirmation.

Pathway E—Community Intelligence Validation Pathway 140e: This pathway cross-references candidate diseases against an anonymized community phenotype intelligence index derived from consented patient-reported data, requiring a configurable minimum number of independent positive references (default: three) before contributing to the ranking. The pathway queries the index for phenotypic similarity between the patient's profile and consented community member profiles associated with each candidate disease, subject to the requesting user's consent tier authorizing use of community intelligence signals.

Before Reciprocal Rank Fusion aggregation, each pathway's ranked output is optionally adjusted by a disease characterization richness prior. For each candidate disease d, a characterization richness score CR(d) is computed from external genomic databases (such as the count of pathogenic and likely pathogenic variant entries in Clin Var associated with genes linked to disease d). The prior adjustment is applied as: S_adjusted(d)=S_pathway(d)+pw×log(1+CR(d)), where pw is a configurable prior weight parameter (default pw=0.1). This prior reflects the empirical observation that diseases with well-characterized genomic associations tend to have more precise phenotype annotations in curated databases, improving the reliability of phenotype-based matching for those diseases. Critically, the characterization richness prior is derived exclusively from external databases (Clin Var, OMIM morbidmap) and never from benchmark evaluation datasets, preventing circular validation.

The consensus aggregation engine 150 implements Reciprocal Rank Fusion (RRF) as the primary aggregation mechanism. For each candidate disease d appearing in one or more pathway-ranked lists, the RRF score is computed as: RRF(d)=Σ[i=1 to N] 1/(k+rank_i(d)), where rank_i(d) is the rank position of disease d in pathway i's ranked list (with rank 1 being the highest-ranked), k is a configurable smoothing parameter (default k=60), and N is the number of active pathways. For diseases not appearing in a pathway's ranked list, a default low rank (default: rank 1000) is assigned.

A consensus bonus B is applied to the RRF score of a candidate disease when a configurable threshold number K of pathway modules assign that disease a rank within a configurable affirmative rank threshold: RRF_final(d)=RRF(d)+B if count(rank_i(d)≤affirmative_rank_threshold)≥K. In a preferred embodiment, B=0.10, K=3, and affirmative_rank_threshold=10.

An insufficient-data response is activated when fewer than two pathway modules produce ranked lists with any candidate diseases, indicating that the phenotype profile contains insufficient information for reliable consensus formation.

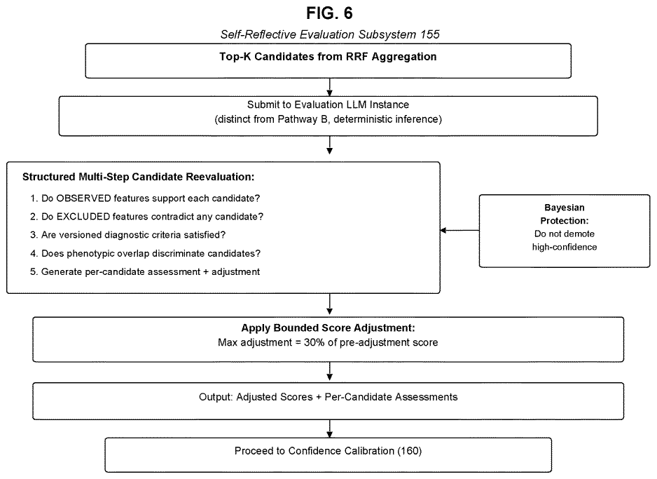

Referring to FIG. 6, the self-reflective evaluation subsystem 155 implements a bounded structured reevaluation process. After initial RRF aggregation, the subsystem conditionally activates when the top-ranked candidate diseases have RRF scores within a configurable ambiguity threshold (default: top-3 candidates within 20% of each other's RRF score). When activated, the subsystem submits the top-K candidates (default K=8) along with the patient's OBSERVED and EXCLUDED HPO feature sets to a large language model evaluation instance configured with: (i) A distinct model instance from Pathway B's LLM, executing under separate constrained inference parameters with temperature set to zero for deterministic evaluation; (ii) An evaluation prompt instructing the model to perform structured multi-step candidate reevaluation, assessing for each candidate disease: (a) whether the patient's OBSERVED features support the diagnosis and which specific features are pathognomonic or highly characteristic; (b) whether any EXCLUDED features contradict the diagnosis by being obligate or very frequent for that disease; (c) whether published diagnostic criteria for the disease, as stored in a versioned guideline reference accessible to the evaluation instance, are satisfied; and (d) whether phenotypic overlap between competing top-ranked candidates provides differential diagnostic discrimination; (iii) A structured output schema requiring the model to emit a ranked reordering of the evaluated candidates with confidence adjustments and per-candidate assessment summaries.

The evaluation output adjusts the RRF_final scores of evaluated candidates within a bounded adjustment range. The bounded adjustment logic operates as follows: FOR each candidate disease d in top-K list: LET adjustment=evaluation_suggested_adjustment(d); IF abs(adjustment)>0.30×RRF_final(d) THEN adjustment=sign(adjustment)×0.30 ×RRF_final(d); END IF; LET second_highest_bayesian=the Bayesian pathway score from pathway module 140a of the candidate having the second-highest such score among the top-K list; IF adjustment<0 AND bayesian_score_140a(d)>second_highest_bayesian×confidence_margin THEN adjustment=0; END IF; RRF_adjusted(d)=RRF_final(d)+adjustment; END FOR. A maximum adjustment magnitude constraint prevents any candidate disease's adjusted score from increasing or decreasing by more than a configurable percentage of its pre-adjustment score (default: 30%). A Bayesian protection constraint prevents the self-reflective subsystem from demoting a candidate whose Bayesian pathway score from pathway module 140a exceeds the second-highest Bayesian pathway score among the top-K candidates by more than a configurable confidence margin (default: 1.5×), protecting high-confidence Bayesian signals from LLM-based override.

In an alternative embodiment, the self-reflective evaluation subsystem 155 operates on all candidate diseases in the top-K list without an ambiguity-threshold gate, performing structured reevaluation on every diagnostic query. This embodiment trades increased computational cost for consistent evaluation quality across all queries.

When the LLM Cross-Validation Pathway 140b or the Self-Reflective Evaluation Subsystem 155 employs a large language model, the system implements temporal stratification analysis to detect potential training data contamination. Cases in the evaluation dataset are stratified into temporal cohorts based on publication date (e.g., pre-2022 and post-2022 relative to the LLM's training data cutoff). If the LLM pathway's contribution to the consensus score shows a statistically significant performance differential between temporal cohorts (assessed via McNemar's test with p<0.05), the system flags potential memorization and reports the performance differential as part of the validation output. This mechanism facilitates transparency and contamination detection in validation workflows for AI-enabled diagnostic systems.

The confidence calibration module 160 classifies each candidate disease into one of four confidence tiers: HIGH (top disease score exceeds second-ranked score by a factor of 2.5 or greater); MODERATE (top-three diseases are within 30% of each other's score); LOW (scores are distributed across many candidates with no clear differentiation); and INSUFFICIENT (fewer than two pathways returned affirmative results). The clinician output module 170 generates a structured differential diagnosis comprising: the top-K ranked candidate diseases with tier classifications; per-disease pathway evidence summaries identifying which pathways contributed to each candidate's ranking; matched and unmatched HPO term annotations for each candidate; per-candidate assessment summaries from the self-reflective evaluation subsystem when activated; a mandatory disclaimer that results are decision support and not a medical diagnosis; recommended next-step actions including specialist referral type, suggested diagnostic tests, and relevant clinical trial identifiers; and source citations for each evidence element. A clinician confirmation gate requires receipt of an explicit electronic acknowledgment signal from an authenticated clinician before the ranked differential is communicated to patient-facing interfaces.

### WORKED EXAMPLE

The following worked example illustrates end-to-end operation of the multi-model consensus diagnostic system 100 on a representative rare disease case. This example is provided for explanatory purposes only; the invention is not limited to this specific case.

Patient Input: A clinician enters the following natural language symptom description for a 6-year-old male patient: “progressive muscle weakness in legs, enlarged calves, difficulty climbing stairs, Gowers sign present, elevated creatine kinase. Patient does NOT have intellectual disability. No cardiac involvement observed.”

Phenotype Normalization Engine 120 Output: The engine maps the input to the following structured phenotype profile. OBSER VED set: HP:0003236 (Elevated circulating creatine kinase), HP:0003325 (Limb-girdle muscle weakness), HP:0003707 (Calf muscle pseudohypertrophy), HP:0002362 (Difficulty climbing stairs), HP:0003391 (Gowers sign). EXCLUDED set: HP:0001249 (Intellectual disability), HP:0001638 (Cardiomyopathy). Confidence scores: all terms mapped at confidence>=0.95 via exact synonym match.

Pathway A (Bayesian, LIRICAL) Output: Rank 1: Duchenne muscular dystrophy (OMIM:310200), posterior probability 0.82. Rank 2: Becker muscular dystrophy (OMIM:300376), posterior probability 0.11. Rank 3: Limb-girdle muscular dystrophy type 2A (OMIM:253600), posterior probability 0.03.

Pathway B (LLM Cross-Validation) Output: The LLM returns structured JSON: [{“disease”: “Duchenne muscular dystrophy”, “confidence”: 0.85}, {“disease”: “Becker muscular dystrophy”, “confidence”: 0.55}, {“disease”: “Emery-Dreifuss muscular dystrophy”, “confidence”: 0.20}]. Multi-tier fuzzy matching resolves all three disease names to valid OMIM identifiers. No null-identifier filtering is triggered in this example because all names resolve successfully.

Pathway C (Frequency-Weighted, Negation-Aware SimGIC) Output: For Duchenne muscular dystrophy: S_simgic=0.72, S_spec=14.8 (unnormalized, dominated by HP:0003707 calf pseudohypertrophy with IC=8.2 and FW=0.95), S_combined=0.25×0.72+0.75×14.8=11.28. Negation penalty: HP:0001249 (intellectual disability) appears in the Duchenne annotation set with frequency “frequent” (IC=3.1), contributing P_neg=3.1/42.5=0.073. HP:0001638 (cardiomyopathy) appears with frequency “occasional” (IC=4.5), contributing additional P_neg. Total P_neg=0.179. S_neg=11.28×(1−0.55×0.179)=10.17. For Becker muscular dystrophy: S_neg=9.42. Rank 1: Duchenne. Rank 2: Becker.

RRF Aggregation: With k=60, Duchenne receives: 1/(60+1)+1/(60+1)+1/(60+1)=0.04918 (rank 1 in all three pathways). Becker receives: 1/(60+2)+1/(60+2)+1/(60+2)=0.04839. Consensus bonus: Duchenne receives B=0.10 (3 pathways agree within top 10). Becker receives B=0.10 (3 pathways agree). RRF_final: Duchenne=0.1492, Becker=0.1484.

Self-Reflective Evaluation (Subsystem 155): Activated because top-2 candidates are within 20% of each other. The evaluation instance performs structured reevaluation: Duchenne is strongly supported by Gowers sign+calf pseudohypertrophy+proximal weakness+elevated CK in a 6-year-old male. The EXCLUDED feature intellectual disability is present in approximately 30% of Duchenne patients but is not obligate, so its absence does not contradict the diagnosis. The EXCLUDED feature cardiomyopathy typically develops later in disease course and its absence at age 6 is expected. Becker is possible but typically presents later with milder course. Adjustment: Duchenne +15% (within 30% bound), Becker −10%. Bayesian protection check: Duchenne Bayesian pathway score from 140a (0.82 ) exceeds the second-highest Bayesian pathway score among the top-K candidates (Becker at 0.11) by more than 1.5×, so the subsystem could not have demoted Duchenne even if the evaluation had suggested it. RRF_adjusted: Duchenne=0.1716, Becker=0.1336.

Confidence Calibration: Score ratio=0.1716/0.1336=1.28. This is below the 2.5 threshold for HIGH, but the top-3 are within 30%, so the tier is classified as MODERATE. Output: Rank 1 Duchenne muscular dystrophy (MODERATE confidence), Rank 2 Becker muscular dystrophy, with per-candidate assessment summaries and pathway evidence. The clinician confirmation gate requires acknowledgment before results are displayed to the patient.

### Null-Identifier Filtering Process

The following describes the null-identifier filtering process applied to LLM pathway outputs before Reciprocal Rank Fusion aggregation: FOR each candidate_name in LLM_output_list: LET matched_id=exact_lookup(normalize(candidate_name), disease_index); IF matched_id is NULL THEN matched_id=edit_distance_match(candidate_name, disease_index, threshold=0.85); END IF; IF matched_id is NULL THEN matched_id=word_overlap_jaccard(candidate_name, disease_index); END IF; IF matched_id is NULL THEN record(candidate_name, identifier=NULL, status=“unresolved”); EXCLUDE candidate_name from RRF_input_list; log_unresolved(candidate_name, source_pathway=“LLM”); ELSE add(candidate_name, matched_id) to RRF_input_list; END IF; END FOR. This filtering prevents hallucinated or misspelled disease names generated by the LLM pathway from corrupting the consensus ranking.

What is claimed is:

1. A computer-implemented system for rare disease differential diagnosis support, comprising: one or more processors and a non-transitory computer-readable medium storing instructions that, when executed by the one or more processors, cause the system to: receive patient phenotypic input comprising descriptions of one or more clinical features; apply a phenotype normalization engine to the patient phenotypic input to generate a structured phenotype profile comprising a set of OBSERVED Human Phenotype Ontology (HPO) identifiers corresponding to features reported as present and a set of EXCLUDED HPO identifiers corresponding to features explicitly reported as absent; concurrently dispatch, via a pathway orchestration module enforcing read-isolation during execution such that no pathway module has read access to the computational state or partial outputs of any other pathway module, the structured phenotype profile to a plurality of heterogeneous diagnostic pathway modules each employing a distinct algorithmic approach; receive, from each diagnostic pathway module, a ranked list of candidate rare diseases, wherein for each candidate identified by a disease name rather than a recognized disease identifier, the system resolves the name against a disease identifier index through multi-tier name matching comprising exact normalized-name lookup, edit-distance-based approximate matching, and word-overlap similarity scoring, and wherein each candidate whose name cannot be resolved to a recognized disease identifier through any tier of matching is recorded with a null identifier and excluded from subsequent rank aggregation; compute, via a consensus aggregation engine implementing Reciprocal Rank Fusion (RRF), an aggregate score for each resolved candidate disease as a sum over pathways of 1/(k+rank_i(d)), wherein k is a configurable smoothing parameter and rank_i(d) is the rank position of the candidate disease in pathway i's ranked list; apply a consensus bonus to the aggregate score of each candidate disease for which the number of pathway modules ranking that disease within a configurable affirmative rank threshold meets or exceeds a configurable pathway agreement threshold; and generate a ranked differential diagnosis with confidence tier classifications and pathway-specific evidence citations, wherein patient-facing communication of the ranked differential diagnosis requires receipt of an explicit clinician confirmation signal.

2. The system of claim 1, wherein the plurality of heterogeneous diagnostic pathway modules comprises at least: a first pathway module applying a Bayesian probabilistic ranking algorithm that computes log-likelihood ratio scores for candidate diseases based on disease-phenotype frequency annotations; a second pathway module executing a large language model under constrained inference parameters comprising a locked model version, a structured JSON output schema, and a temperature parameter set to zero; and a third pathway module computing a negation-aware information content-weighted phenotypic similarity score.

3. The system of claim 2, wherein the third pathway module computes a frequency-weighted specificity score defined as: S_spec(q,d)=Σ(FW(t,d)×IC(t)), where the summation is over all HPO terms t present in both the OBSERVED identifier set Q_obs and the disease annotation set D, wherein FW(t,d) is a frequency weight for HPO term t derived from curated disease-phenotype frequency annotations for disease d, and IC(t) is the information content of term t, and wherein the specificity score is computed as an unnormalized sum without division by a union-based denominator.

4. The system of claim 3, wherein the third pathway module combines the frequency-weighted specificity score with a standard SimGIC similarity score via a configurable weighting parameter w according to: S_combined(q,d)=(1−w)×S_simgic(q,d)+w×S_spec(q,d), and further computes a negation penalty P_neg for EXCLUDED HPO features, yielding a final score: S_neg(q,d)=S_combined(q,d)×(1−λ×P_neg), wherein λ is a configurable penalty weight parameter and P_neg is computed as the ratio of the sum of information content of EXCLUDED HPO terms appearing in the disease annotation set to the total information content of all terms in the disease annotation set.

5. The system of claim 2, further comprising a self-reflective evaluation subsystem configured to apply bounded score adjustments to top-ranked candidate diseases, wherein the bounded score adjustments are constrained by a Bayesian protection constraint that prevents demotion of a candidate whose Bayesian pathway score from the first pathway module exceeds the highest Bayesian pathway score among all other candidates in the top-K list from the first pathway module by more than a configurable confidence margin.

6. The system of claim 2, wherein the third pathway module further expands the OBSERVED HPO identifier set by incorporating ancestor terms from the HPO ontology hierarchy with a reduced weight coefficient, enabling matching against diseases annotated with broader phenotypic terms when specific terms are not present in the disease annotation database.

7. The system of claim 2, wherein when a diagnostic pathway module produces a candidate disease name that cannot be matched to a recognized disease identifier through exact normalized-name lookup, edit-distance-based approximate matching, or word-overlap similarity scoring, the candidate is recorded with a null identifier and excluded from the Reciprocal Rank Fusion aggregation, preventing unresolvable disease names from affecting the consensus ranking.

8. The system of claim 1, further comprising a self-reflective evaluation subsystem configured to, upon activation: select the top-K candidate diseases from the ranked differential diagnosis; retrieve, from a versioned guideline reference, published diagnostic criteria applicable to each of the top-K candidate diseases; generate, for each candidate, a structured assessment comprising an observed-feature support determination, an excluded-feature contradiction determination, and a guideline-criteria satisfaction determination; and apply a bounded score adjustment to each candidate, the adjustment constrained within a configurable maximum adjustment magnitude.

9. The system of claim 1, wherein the pathway orchestration module enforces pathway independence by executing all diagnostic pathway modules simultaneously via asynchronous parallel execution, wherein no pathway module has read access to the computational state or partial outputs of any other pathway module during execution.

10. The system of claim 1, further comprising a disease characterization richness prior subsystem that adjusts each pathway's ranked output by a prior score CR(d) derived from external gene-disease association databases, wherein the prior adjustment is computed as S_adjusted(d)=S_pathway(d)+pw×log(1+CR(d)), wherein pw is a configurable prior weight parameter and CR(d) is a count of pathogenic variant entries in external databases associated with genes linked to disease d.

11. The system of claim 1, further comprising a community intelligence validation pathway that cross-references candidate diseases against an anonymized community phenotype intelligence index populated from consented patient-reported data, wherein activation of the community intelligence validation pathway is gated by the requesting user's consent tier assignment, and wherein a configurable minimum number of independent positive references is required before the community signal contributes to the ranking.

12. The system of claim 1, wherein the system activates an insufficient-data response path when fewer than two diagnostic pathway modules produce ranked lists with any candidate diseases, the insufficient-data response path generating a specialist referral recommendation in lieu of a ranked differential diagnosis.

13. The system of claim 1, further comprising a temporal stratification subsystem that stratifies evaluation cases by publication date relative to a large language model training data cutoff and reports a performance differential metric between temporal cohorts, enabling detection of LLM training data contamination.

14. The system of claim 1, wherein the plurality of heterogeneous diagnostic pathway modules further comprises a genetic variant prioritization pathway conditionally activated when genomic variant data is present, the genetic variant prioritization pathway applying: variant pathogenicity classification scoring weighting pathogenic variants higher than variants of uncertain significance; inheritance pattern filtering requiring genotype consistency with disease inheritance mode; and variant type scoring differentiating between loss-of-function variants and missense variants, wherein loss-of-function variants receive a higher pathogenicity weight than missense variants.

15. The system of claim 1, wherein the patient phenotypic input is received via one or more of: typed natural language text, structured checklist selections mapped to HPO term identifiers, voice transcription output from a speech-to-text engine configured with medical vocabulary models, structured clinical documents including clinical notes and genetic testing reports, or a multi-modal combination of two or more of the foregoing modalities.

16. A computer-implemented method for rare disease differential diagnosis support, comprising: generating a structured phenotype profile comprising OBSERVED and EXCLUDED Human Phenotype Ontology (HPO) identifier sets from patient phenotypic input; concurrently executing, in parallel and under read-isolation preventing intermediate result sharing, a plurality of heterogeneous diagnostic pathway modules against the structured phenotype profile, each module employing a distinct algorithmic approach, wherein at least one pathway module computes a frequency-weighted, negation-aware phenotypic similarity score for each candidate disease by: (i) computing a frequency-weighted specificity score as an unnormalized sum of frequency-weighted information content values for OBSERVED HPO terms matching the disease's annotation profile; and (ii) penalizing candidates whose annotated phenotype profiles include HPO features present in the EXCLUDED set, the penalty weighted by the information content of each excluded feature within the candidate disease's annotated phenotype profile; for each candidate identified by a disease name rather than a recognized disease identifier, resolving the name through multi-tier name matching and excluding each candidate whose name cannot be resolved from rank aggregation; aggregating ranked lists produced by each pathway module using Reciprocal Rank Fusion to compute aggregate scores and applying a consensus bonus to candidate diseases receiving affirmative rankings from a threshold number of pathway modules; applying a bounded self-reflective evaluation to ambiguous top-ranked candidates using a large language model evaluation instance that performs structured multi-step candidate reevaluation with bounded score adjustment constraints and Bayesian protection preventing demotion of high-confidence candidates; and generating a confidence-tiered ranked differential diagnosis with pathway-specific evidence citations, requiring clinician confirmation before patient-facing communication.

17. The method of claim 16, further comprising adjusting pathway scores by a disease characterization richness prior derived from external gene-disease association databases before Reciprocal Rank Fusion aggregation.

18. The method of claim 16, further comprising activating an insufficient-data response path when fewer than two pathway modules produce ranked lists with candidate diseases.

19. The method of claim 16, wherein aggregating comprises computing RRF(d)=Σ1/(k+rank_i(d)) for each candidate disease d across N pathway modules, wherein k is a configurable parameter in the range of 30 to 100.

20. The method of claim 16, further comprising performing temporal stratification analysis on evaluation results to detect large language model training data contamination by comparing performance metrics between temporal cohorts.

21. A non-transitory computer-readable medium storing instructions that, when executed by one or more processors, cause a system to: normalize patient phenotypic input to a structured phenotype profile comprising OBSERVED and EXCLUDED Human Phenotype Ontology identifier sets; execute a plurality of heterogeneous diagnostic pathways concurrently under read-isolation preventing intermediate result sharing, wherein at least one pathway applies frequency-weighted, negation-aware scoring that: (i) computes an unnormalized frequency-weighted specificity score reflecting curated disease-phenotype frequency annotations; and (ii) penalizes candidate diseases based on information content of EXCLUDED HPO features appearing in each disease's phenotype annotation profile, each pathway producing an independently derived ranked list of rare disease candidates; for each candidate identified by a disease name rather than a recognized disease identifier, resolve the name through multi-tier name matching and exclude each candidate whose name cannot be resolved from rank aggregation; adjust pathway scores by a disease characterization richness prior derived from external gene-disease association databases; aggregate ranked lists via Reciprocal Rank Fusion with a consensus bonus for inter-pathway agreement; apply a bounded self-reflective evaluation with structured multi-step candidate reevaluation and bounded adjustment constraints including Bayesian protection to top-ranked candidates; and generate a confidence-tiered differential diagnosis requiring clinician confirmation before patient communication.
